(19) United States
(12) Patent Application Publication
(10) Pub. No.: US 2010/0222860 A1
(43) Pub. Date: Sep. 2, 2010

(54) Implantable Leads Having Mechanisms to Impede Over-Rotation of Fixation Mechanisms

(75) Inventors: Christina Casella, Los Angeles, CA (US); Rolf Hill, Jarfalla (SE); Per Jarl, Jarfalla (SE)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/396,299

(22) Filed: Mar. 2, 2009

(51) Int. Cl. A61N 1/05

(52) U.S. Cl. 607/127

(57) ABSTRACT

An implantable lead includes a lead body, a header body, a fixation mechanism, a rotatable shaft and a rotation limit element. The fixation mechanism is disposed in the header body and is extendable out of the header body for securing the header body to cardiac tissue of the patient. The shaft is provided in the header body and is coupled to the fixation mechanism for translating rotational movement of the shaft into linear displacement of the fixation mechanism. The rotation limit element is disposed in the header body. The rotation limit element engages the header body once the shaft is linearly displaced by a predetermined distance with respect to the header body to prevent further rotation of the shaft and limit additional displacement of the fixation mechanism with respect to the header body.

### FIELD OF THE INVENTION

The various embodiments described herein generally relate to implantable leads, and more particularly to implantable leads having extendable and retractable fixation mechanisms.

### BACKGROUND OF THE INVENTION

An implantable medical device is implanted in a patient to, among other things, monitor electrical activity of a heart and to deliver appropriate electrical therapy as required. Implantable medical devices (“IMDs”) include for example, pacemakers, cardioverters, defibrillators, implantable cardioverter defibrillators (“ICD”), and the like. The electrical therapy produced by an IMD may include, for example, pacing pulses, cardioverting pulses, and/or defibrillator pulses to reverse arrhythmias (e.g. tachycardias and bradycardias) or to stimulate the contraction of cardiac tissue (e.g. cardiac pacing) to return the heart to its normal sinus rhythm. The devices include leads that are implanted to cardiac tissue to monitor the activity of the heart and to deliver the therapy to the heard. The leads are secured to the cardiac tissue using fixation helices that extend from and retract into the distal end of the lead. The fixation helices puncture the cardiac tissue to secure the lead to the tissue.

Known fixation helices are extended from and retracted into the leads by rotating a connector pin at the proximal end of the lead. The connector pin is interconnected with a rotatable shaft in the lead by a conductor. Rotation of the connector pin also rotates the shaft within the lead. The outer surface of the shaft and the inner diameter of the distal end of the lead include threaded surfaces that engage one another. The threaded surfaces engage each other to translate the rotation of the shaft into displacement of the shaft within the lead. The shaft also is coupled with the fixation helix of the lead. As the shaft is rotated and linearly displaced, the fixation helix also is rotated and linearly displaced. Therefore, to extend and retract the fixation helix from the lead, the connector pin is rotated to cause rotation and displacement of the shaft and fixation helix. In other known leads, an inwardly protruding post in the distal end of the lead engages a fixation helix that is wound in a spiral. As the fixation helix is rotated, the fixation helix slides along the post as the post remains stationary with respect to the lead. The movement of the fixation helix with respect to the post causes the fixation helix to be extended from or retracted into the lead, depending on the direction of rotation and the direction in which the fixation helix is wound.

The extension of fixation helices in known leads is limited to prevent the fixation helix from protruding too far into the cardiac tissue and causing significant damage to the heart. During translation of the fixation helix in the lead, the shaft contacts the inside surface of the lead to prevent further linear displacement of the helix with respect to the lead. For example, the inside surface of the lead may include a pair of shoulders at the interfaces between different inside diameters. As the shaft is extended through a larger inside diameter of the lead, the helix is extended along a longitudinal axis of the lead. When the shaft is extended to the shoulder at the smaller inside diameter, the shaft is prevented from being extended any further in the lead. Similarly, as the shaft is retracted from a larger inside diameter to a smaller inside diameter of the lead, the shaft may engage another shoulder in the lead to prevent the shaft from being retracted any further in the lead.

But, the engagement between the shaft and the inside surfaces of the lead does not prevent the shaft from continuing to rotate. When the shaft includes a threaded surface that engages a threaded surface in the lead to displace the shaft and fixation helix, continued rotation of the shaft will not result in continued linear displacement of the shaft. This rotation with no linear displacement may cause an over-torque or tightening of the threaded engagement between the shaft and the lead. If the shaft includes an inner post that is engaged by the fixation helix, continued rotation of the shaft will not result in continued linear displacement of the shaft but may cause the fixation helix to become deformed. For example, the helix may build up between the helix and header posts.

The tightening of the threaded connection and the building up of the helix may cause the fixation helix to abruptly move, or jump, when the connector pin and shaft are rotated in the opposing direction of the over-torque. The jumping of the helix may cause unnecessary damage to the cardiac tissue. Alternatively, the helix may become stuck or locked in an extended or retracted state and unable to be easily retracted or extended from the locked state. Thus, a need exists for a lead that permits the extension and retraction of a fixation mechanism while preventing damage or over-torque to the internal components of the lead that extend and retract the fixation mechanism.

### BRIEF SUMMARY OF THE INVENTION

In one embodiment, an implantable lead is provided. The lead includes a lead body, a header body, a fixation mechanism, a rotatable shaft and a rotation limit element. The lead body extends between a distal end and a proximal end and is configured to be implanted in a patient. The header body is joined to the lead body at the distal end of the lead body. The fixation mechanism is disposed in the header body and is extendable out of the header body for securing the header body to cardiac tissue of the patient. The shaft is provided in the header body and is coupled to the fixation mechanism for translating rotational movement of the shaft into linear displacement of the fixation mechanism. The rotation limit element is disposed in the header body. The rotation limit element engages the header body once the shaft is linearly displaced by a predetermined distance with respect to the header body to prevent further rotation of the shaft and limit additional displacement of the fixation mechanism with respect to the header body.

In another embodiment, another implantable lead is provided. The lead includes a lead body, a header body, a fixation mechanism and a rotatable shaft. The lead body extends between a distal end and a proximal end and is configured to be implanted in a patient. The header body is joined to the lead body at the distal end of the lead body. The fixation mechanism is disposed in the header body and is extendable out of the header body for securing the header body to cardiac tissue of the patient. The shaft is provided in the header body and is coupled to the fixation mechanism for translating rotational movement of the shaft into linear displacement of the fixation mechanism. The shaft engages the header body once the shaft is linearly displaced by a predetermined distance with respect to the header body to prevent further rotation of the shaft and to limit additional linear displacement of the fixation mechanism with respect to the header body.

In another embodiment, an implantable lead is provided. The lead includes a lead body, a header body, a fixation mechanism, and a rotatable shaft. The lead body extends between a distal end and a proximal end and is configured to be implanted in a patient. The header body is joined to the lead body at the distal end of the lead body. The fixation mechanism is disposed in the header body and is configured to secure the header body to cardiac tissue of the patient. The shaft is provided in the header body and is coupled to the fixation mechanism. The shaft translates rotational movement of the shaft into linear displacement of the fixation mechanism to extend and retract the fixation mechanism with respect to the header body. The shaft engages the header body once the shaft is linearly displaced by at least one of a predetermined extension distance and a predetermined retraction distance with respect to the header body to prevent further rotation of the shaft and limit additional displacement of the fixation mechanism with respect to the header body.

### BRIEF DESCRIPTION OF THE DRAWINGS

The drawings illustrate generally, by way of example, but not by way of limitation, various embodiments discussed in the present document.

FIG. 1 illustrates an implantable medical system formed in accordance with one example embodiment.

FIG. 2 illustrates the lead shown in FIG. 1.

FIG. 3 is a cross-sectional view of a header body of the lead shown in FIG. 1 with a fixation mechanism in a retracted position in accordance with one embodiment.

FIG. 4 is a cut-away view of the header body shown in FIG. 2 that is implemented in accordance with one embodiment.

FIG. 5 is a cross-sectional view of the header body shown in FIG. 2 with the fixation mechanism also shown in FIG. 2 in an extended position in accordance with one embodiment.

FIG. 6 is a cut-away view of the header body shown in FIG. 2 that is implemented in accordance with one embodiment.

FIG. 7 is a cross-sectional view of a header body in accordance with an alternative embodiment.

FIG. 8 is a partial sectional view of a proximal end of the header body shown in FIG. 7 in accordance with one embodiment.

FIG. 9 is a perspective view of a rotatable shaft joined to a fixation mechanism in accordance with one embodiment.

FIG. 10 is a partial sectional view of a proximal end of a header body in accordance with another embodiment.

### DETAILED DESCRIPTION OF THE INVENTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. For example, embodiments may be used with a pacemaker, a cardioverter, a defibrillator, and the like. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated.

In accordance with certain embodiments, implantable leads are provided that impede over-rotation of the fixation mechanisms in the leads. The leads include structures for stopping the continued rotation of the fixation mechanisms after the fixation mechanisms are extended out of the lead and after the fixation mechanisms are retracted into the lead. Preventing over-rotation of the fixation mechanisms may prevent over-torquing the mechanisms and shafts to which the mechanisms are connected. Reducing the over-torque on the fixation mechanisms and shafts may prevent the mechanisms from abruptly moving, or “jumping,” when the mechanisms are extended toward or retracted away from the cardiac tissue.

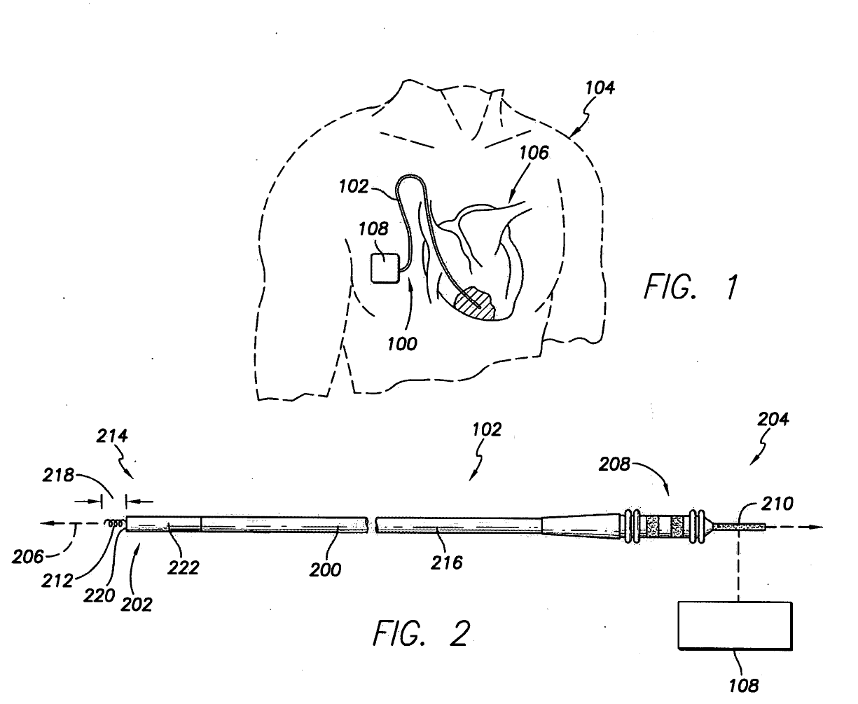

FIG. 1 illustrates an implantable medical system 100 including an implantable lead 102 formed in accordance with one example embodiment. FIG. 1 depicts a chest cavity 104 in phantom, and a heart 106 within the chest cavity 104. The medical system 100 includes an implantable medical device 108, such as a pacemaker, and the lead 102, which are both implanted in the chest cavity 104. Optionally, the medical device 108 may be implanted in a position other than the position shown in FIG. 1. In the illustrated embodiment, the lead 102 is a bipolar pacing and sensing lead, however other types of leads may be used.

FIG. 2 illustrates the lead 102 in more detail. The lead 102 has an elongated body 200 that includes a distal end portion 202 and a proximal end portion 204. The lead body 200 has a length that extends along a longitudinal axis 206 between the distal and proximal end portions 202, 204. The longitudinal axis 206 may be a linear or a non-linear axis. The longitudinal axis 206 of the lead body 200 extends along a curved path that changes as the lead body 200 is flexed, bent and otherwise manipulated.

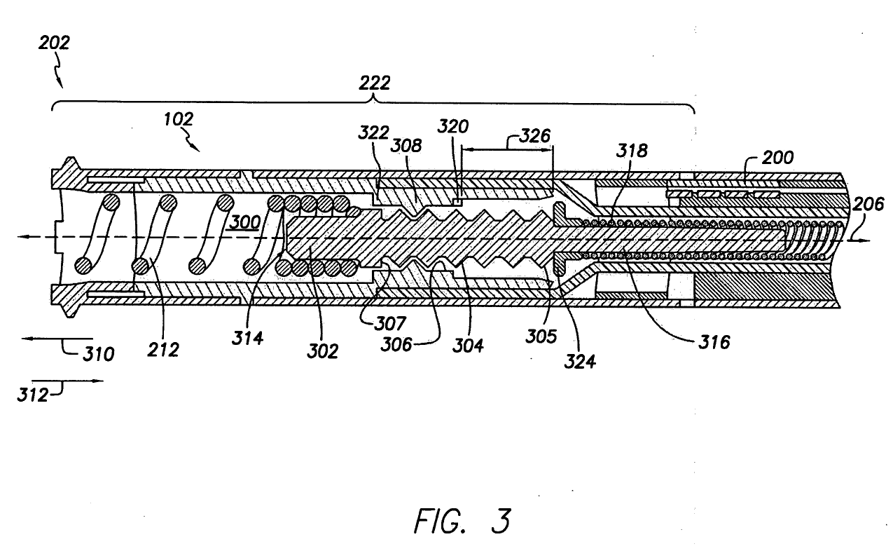

A connector assembly 208 is provided at the proximal end portion 204 of the lead 102. The connector assembly 208 is configured to be inserted into a receiving orifice in the implantable medical device 108 (shown in FIG. 1). The connector assembly 208 includes at least one electrical terminal 210 that is connected to an electrical conductor 318 (shown in FIG. 3). The conductor 318 is enclosed by an insulative sheath 216 that extends along the lead body 200. An electrode assembly 214 is provided at the distal end portion 202 of the lead 102. The electrode assembly 214 is electrically coupled with the medical device 108 via the conductor 318 and the connector assembly 208.

The electrode assembly 214 includes a header body 222 that at least partially houses a fixation mechanism 212. The fixation mechanism 212 is electrically connected to the electrical terminal 210 by the conductor 318 (shown in FIG. 3). Alternatively, an electrode separate from the fixation mechanism 212 may be provided that is electrically coupled with the terminal 210 by the conductor 318. The fixation mechanism 212 functions to interlock with cardiac tissue at an implantation site and thereby prevent inadvertent displacement of the distal end portion 202 of the lead 102 once the lead 102 is implanted. The fixation mechanism 212 is moveable in opposing directions along the longitudinal axis 206 in order to retract and extend the fixation mechanism 212 into and away from the header body 222. The fixation mechanism 212 is extended from the header body 222 to an extended position. In the extended position, the fixation mechanism 212 protrudes from an end tip 220 of the header body 222 by a predetermined extension distance 218. The extension distance 218 may be established to be sufficiently great to secure the distal end portion 202 of the lead 102 to cardiac tissue. Additionally, the extension distance 218 may be established to prevent the fixation mechanism 212 from penetrating too far into the cardiac tissue. In the illustrated embodiment, the fixation mechanism 212 is represented by a screw-in helix that penetrates the cardiac tissue to anchor the lead 102 thereto. While the helix represents one type of fixation mechanism 212, optionally other fixation mechanisms may be used to position and secure the distal end portion 202 to cardiac tissue of a patient.

FIG. 3 is a cross-sectional view of the header body 222 of the lead 102 with the fixation mechanism 212 in a retracted position in accordance with one embodiment. The fixation mechanism 212 is disposed in the header body 222. In the retracted position, the fixation mechanism 212 is at least partially retracted into the header body 222. For example, in the retracted position, the fixation mechanism 212 may be retracted into the header body 222 such that the fixation mechanism 212 is entirely enclosed within the header body 222. The fixation mechanism 212 is coupled to a distal end 314 of a rotatable shaft 302 in the header body 222. The shaft 302 may include, or be formed of, a conductive material. The fixation mechanism 212 may be electrically joined to the shaft 302 to provide a conductive pathway between the fixation mechanism 212 and the shaft 302.

The shaft 302 includes a threaded outer surface 304 having inner and outer ends 305, 307. An inside surface 306 of the header body 222 includes inwardly protruding threads 308. The inwardly protruding threads 308 engage the threaded surface 304 of the shaft 302 to translate rotation of the shaft 302 into linear displacement of the shaft 302. For example, rotation of the shaft 302 in a clockwise direction may linearly displace the shaft 302 within the header body 222 in an extension direction 310 along the longitudinal axis 206. Rotation of the shaft 302 in the counter-clockwise direction linearly displaces the shaft 302 in the header body 222 in an opposing retraction direction 312.

The shaft 302 is coupled to the electrical conductor 318 that extends through the lead 102 shown in FIG. 1. The conductor 318 may be wound in a coil that is joined to a proximal end 316 of the shaft 302. The shaft 302 and conductor 318 are mechanically coupled with one another such that rotation of the conductor 318 during implantation or removal of the lead 102 into the chest cavity 104 (shown in FIG. 1) also rotates the shaft 302. For example, the conductor 318 may be coupled to a connector pin (not shown) in, at or near the proximal end portion 204 (shown in FIG. 2) of the lead 102. Rotation of the connector pin also rotates the conductor 318. The shaft 302 translates rotation of the conductor 318 into linear displacement of the fixation mechanism 212 along the extension and retraction directions 310, 312. The shaft 302 and conductor 318 are electrically connected with one another such that a conductive pathway is provided between the conductor 318 and the shaft 302. The shaft 302 provides an electrically conductive pathway between the conductor 318 and the fixation mechanism 212 such that the fixation mechanism 212 may communicate sensed cardiac signals to the implantable medical device 108 (shown in FIG. 1) and/or apply stimulation pulses generated in the implantable medical device 108 to cardiac tissue.

The header body 222 includes proximal and distal posts 320, 322 that protrude inwardly into the interior cavity 300 of the header body 222 from the inside surface 306. The proximal post 320 is disposed along the longitudinal axis 206 of the header body 222 between the inwardly protruding thread 308 and an inner end of the shaft 302 coupled to the conductor 318. More than one proximal post 320 may be included in the header body 222. The distal post 322 is disposed between the thread 308 and the fixation mechanism 212. More than one distal post 322 may be provided in the header body 222. The header body 222 includes a rotation limit element 324 that is joined to the shaft 302. The rotation limit element 324 is coupled to the shaft 302 and is positioned along the length of the shaft 302 proximate to an end of the conductor 318. The rotation limit element 324 is located along the length of the shaft 302 inward from the conductor 318. As the shaft 302 is rotated to linearly displace the shaft 302 and the fixation mechanism 212 with respect to the header body 222, the rotation limit element 324 also linearly travels at a position along the shaft 302 to be spaced in the extension direction 310.

The rotation limit element 324 is located a predetermined extension distance 326 from the proximal post 320 when the fixation mechanism 212 is in the retracted state shown in FIG. 3. The extension distance 326 may be measured in a direction parallel to the longitudinal axis 206. The extension distance 326 represents the distance that the fixation mechanism 212 may travel in the extension direction 310 along the longitudinal axis 206 before the rotation limit element 324 engages the proximal post 320. Alternatively, the extension distance 326 may represent the predetermined maximum distance that the fixation mechanism 212 may be linearly extended to project from the header body 222 and secure the lead 102 to cardiac tissue. For example, the extension distances 218 (shown in FIG. 2) and 326 (shown in FIG. 3) may be approximately the same. The rotation limit element 324 engages the proximal post 320 to impede or stop continued rotation of the rotation limit element 324 once the fixation mechanism 212 has traveled along the extension distance 326. When the proximal post 320 contacts the rotation limit element 324, the proximal post 320 prevents the rotation limit element 324 and the shaft 302 from continuing to rotate in one rotation direction. For example, the proximal post 320 may prevent the rotation limit element 324 and shaft 302 from continuing to be rotated in the direction that translates the shaft 302 in the extension direction 310 but permit rotation of the shaft 302 in the opposing direction in order to retract the shaft 302 and fixation mechanism 212. Stopping the continued rotation of the shaft 302 may prevent the shaft 302 from continuing to torque the fixation mechanism 212 once the shaft 302 projects a predetermined maximum safe distance from the header body 222.

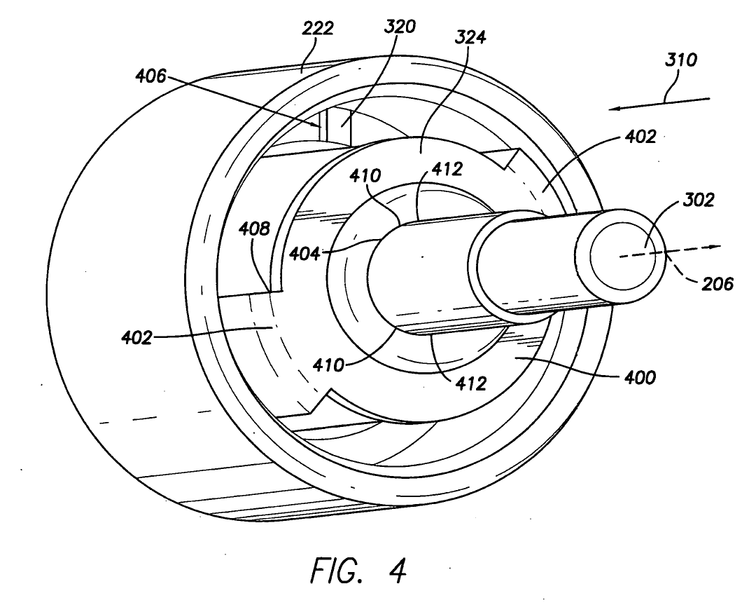

FIG. 4 is a cut-away view of the header body 222 that is implemented in accordance with one embodiment. As shown in FIG. 4, the rotation limit element 324 includes an approximately circular body 400 with radially projecting flanges 402. A different number of flanges 402 may be provided. The flanges 402 may be provided on opposing sides of the body 400. For example, the flanges 402 may be provided approximately 180 degrees apart from one another along the outer perimeter of the body 400. Optionally, the flanges 402 may be disposed different distances from one another. The flanges 402 include sides 406 that engage sides 408 of the proximal post 320. For example, as the shaft 302 and rotation limit element 324 are rotated and displaced in the extension direction 310, the rotation limit element 324 rotates and is translated in the extension direction 310 until the side 408 of one of the flanges 402 engages the side 406 of the proximal post 320. In another embodiment, the sides 408 of two or more flanges 402 may engage the sides 406 of two or more proximal posts 320 at approximately the same time. The engagement between the rotation limit element 324 and the side 406 of the proximal post 320 stops the continued rotation of the rotation limit element 324 and the shaft 302 once the fixation mechanism 212 (shown in FIG. 2) is displaced by the extension distance 326 (shown in FIG. 3).

The flanges 402 may be disposed approximately 180 degrees apart from one another to avoid misaligning the rotation limit element 324 with respect to the shaft 302. Using multiple flanges 402 avoids the risk of assembling the shaft 302 and rotation limit element 324 out of phase with the proximal post 320. For example, when the rotation limit element 324 is mounted onto the shaft 302, the rotation limit element 324 may be mounted such that the flanges 402 are positioned on opposite sides of the proximal post 320 along the longitudinal axis 206. As the rotation limit element 324 rotates and approaches the proximal post 320, one of the multiple flanges 402 engages the post 320 before the fixation mechanism 212 extends too far from the header body 222. If only a single flange 402 were provided, assembling the rotation limit element 324 may require alignment of the flange 402 on the proper side of the proximal post 320 to avoid permitting the fixation mechanism 212 from extending too far from the header body 222 before the single flange 402 engages the proximal post 320.

The circular body 400 includes an opening 404 extending through the body 400. The shaft 302 extends through the opening 404 to couple the shaft 302 and rotation limit element 324 together. The opening 404 and shaft 302 may include polarization features 410, 412 to align the rotation limit element 324 with respect to the shaft 302. In the illustrated embodiment, the polarization features 410 are flat edges of the opening 404 and the polarization feature 412 are flat surface portions of the shaft 302. For example, aligning the polarization features 410, 412 with one another may align the flanges 402 of the rotation limit element 324 with the proximal post 320 such that the flanges 402 engage the proximal post 320 when the shaft 302 has been displaced by the extension distance 326 and not before or after the shaft 302 is displaced by the extension distance 326.

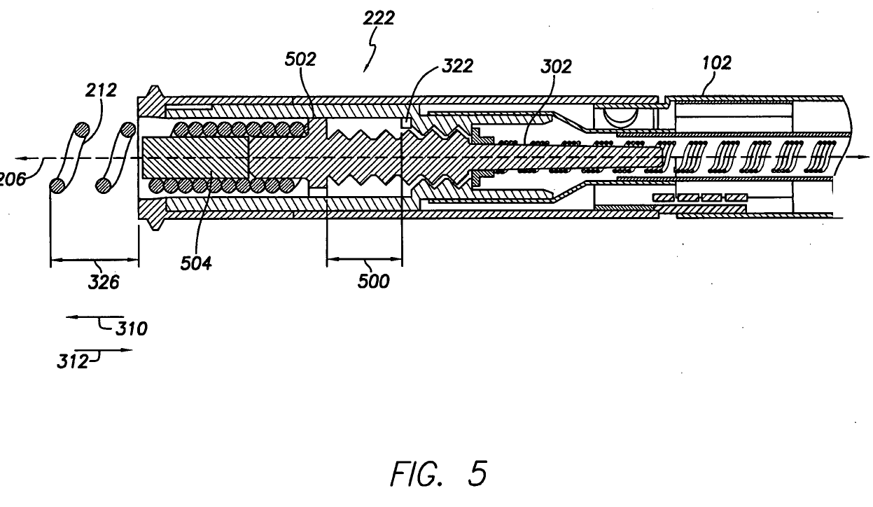

FIG. 5 is a cross-sectional view of the header body 222 of the lead 102 with the fixation mechanism 212 in an extended position in accordance with one embodiment. In the extended position, the fixation mechanism 212 is linearly displaced by the extension distance 326 in the extension direction 310 along the longitudinal axis 206. In order to retract the fixation mechanism 212 into the header body 222, the shaft 302 is rotated in an opposite direction than the shaft 302 is rotated to extend the fixation mechanism 212 out of the header body 222. As the shaft 302 is rotated to retract the fixation mechanism 212, shaft 302 moves in the retraction direction 312 until the shaft 302 engages the distal post 322.

The shaft 302 includes at least one outwardly projecting flange 502 that engages the distal post 322 when the fixation mechanism 212 is retracted into the header body 222. The flange 502 of the shaft 302 is located a predetermined retraction distance 500 along the longitudinal axis 206 from the distal post 322 when the fixation mechanism 212 is in the extended position. The retraction distance 500 may be approximately the same distance as the extension distance 326 (shown in FIG. 3). Alternatively, the retraction and extension distances 500, 326 may differ from one another. The retraction distance 500 may be preset or established to permit the fixation mechanism 212 to be retracted within the header body 222 such that the fixation mechanism 212 is detached from the cardiac tissue and housed within the header body 222.

The engagement between the distal post 322 and the flange 502 of the shaft 302 may prevent the shaft 302 from continuing to be rotated in a direction that retracts the shaft 302 and the fixation mechanism 212. Stopping the continued rotation of the shaft 302 may prevent the shaft 302 from continuing to torque the shaft 302 once the fixation mechanism 212 is retracted away from cardiac tissue and into the header body 222. The engagement between the shaft 302 and the distal post 322 may permit rotation of the shaft 302 in the opposing direction. For example, the shaft 302 may still be rotated in an opposing direction to extend the fixation mechanism 212 out of the header body 222.

In the illustrated embodiment, a medicinal plug 504 is provided in the header body 222 proximate the distal end of the shaft 302. The medicinal plug 504 may enclose or hold one or more drugs useful in the implantation of the fixation mechanism 212 into cardiac tissue. The medicinal plug 504 travels along the longitudinal axis 206 when the shaft 302 moves along the longitudinal axis 206. The shaft 302 pushes the medicinal plug 504 toward the cardiac tissue in order to deliver a drug to the cardiac tissue. For example, the medicinal plug 504 may be a steroid plug that delivers a predetermined dose of a steroid to the cardiac tissue when the fixation mechanism 212 is implanted into the cardiac tissue.

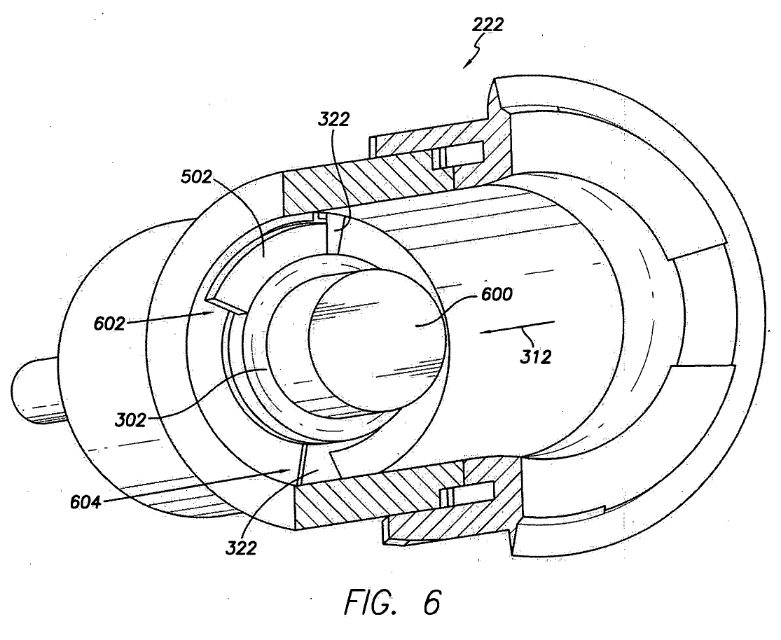

FIG. 6 is a cut-away view of the header body 222 that is implemented in accordance with one embodiment. As shown in FIG. 6, the flange 502 of the shaft 302 radially projects from the shaft 302. The shaft 302 includes an approximately tubular body 600. A different number of flanges 502 may outwardly project from the shaft 302. The flange 502 includes a side 602 that engages a side 604 of the distal post 322. In the illustrated embodiment, the header body 222 includes two opposing distal posts 322, although a different number may be provided. As the shaft 302 and flange 502 are rotated and displaced in the retraction direction 312, the flange 502 moves in the retraction direction 312 until the side 602 engages the side 604 of the distal post 322. In another embodiment, the sides 602 of two or more flanges 502 may engage the sides 604 of two or more distal posts 322 at approximately the same time. The engagement between the flange 502 and the distal post 322 stops the continued rotation of the shaft 302.

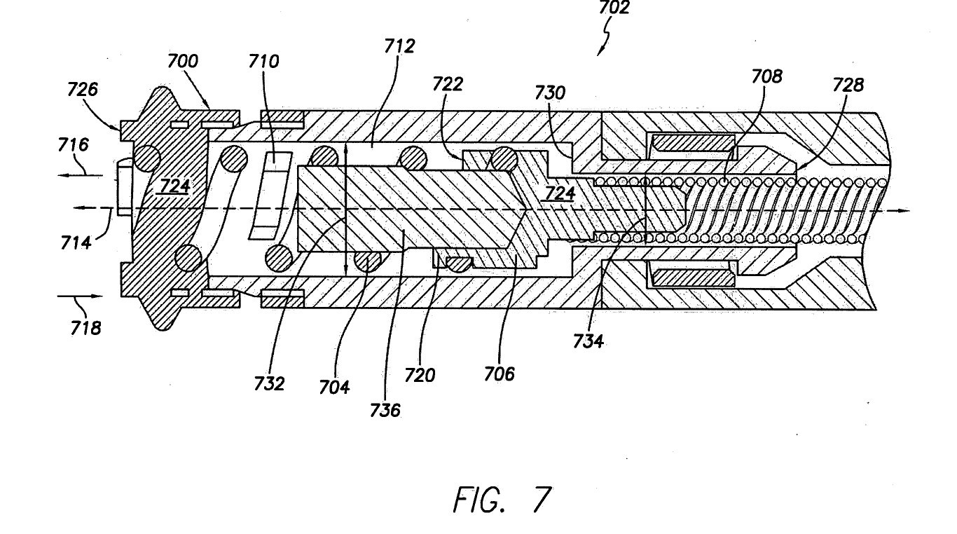

FIG. 7 is a cross-sectional view of a header body 700 of a lead 702 in accordance with an alternative embodiment. The header body 700 and lead 702 are similar to the header body 222 (shown in FIG. 2) and lead 102 (shown in FIG. 1) described above. The header body 700 may enclose a medicinal plug 736 that is similar to the medicinal plug 504 (shown in FIG. 5). The header body 700 includes an interior chamber 724 that extends from proximate a tip 726 of the lead 702 to an inner end 728 located within the lead 702. The header body 700 includes an interior shoulder 730 along the inner surface of the header body 700 and located at the interface of two inner diameters 732, 734 of the interior chamber 724.

A fixation mechanism 704 that is similar to the fixation mechanism 212 (shown in FIG. 2) is disposed in the interior chamber 724. The fixation mechanism 704 is joined to a rotatable shaft 706. As shown in FIG. 7, the shaft 706 includes a helical channel 724 extending around the outer surface of the shaft 706. The fixation mechanism 704 is received in the channel 724 to secure the fixation mechanism 704 to the shaft 706. The rotatable shaft 706 is coupled with an electrical conductor 708 that is similar to the electrical conductor 318 (shown in FIG. 3). The shaft 706 and fixation mechanism 704 may include, or be formed from, conductive materials in order to provide an electrically conductive pathway that extends from the fixation mechanism 704 to the conductor 708. The header body 700 includes a band 710 that protrudes inward from an inside surface 712 of the header body 700. The band 710 may be embodied in a post, marker band, block, or other structural element that extends inward from the header body 700.

The shaft 706 is rotated in each of clockwise and counter-clockwise directions to rotate the fixation mechanism 704 in a similar direction. The fixation mechanism 704 engages the band 710 as the fixation mechanism 704 is rotated. The fixation mechanism 704 slides along the band 710 to translate the rotation of the fixation mechanism 704 into linear displacement of the shaft 706 and the fixation mechanism 704 along a longitudinal axis 714 of the lead 702. For example, rotation of the fixation mechanism 704 in a clockwise direction may cause the fixation mechanism 704 and shaft 706 to travel along the longitudinal axis 714 in an extension direction 716 while rotation of the fixation mechanism 704 in a counter-clockwise direction may retract the fixation mechanism 704 and shaft 706 along an opposite retraction direction 718.

In the illustrated embodiment, the shaft 706 includes a distal rotation limit element 720 proximate a distal end 722 of the shaft 706. The rotation limit element 720 includes a tab that extends along the length of the shaft 706 along the extension direction 716 past the distal end 722. The rotation limit element 720 is shaped and positioned to engage the band 710 of the header body 700 when the fixation mechanism 704 travels in the extension direction 716 by a predetermined extension distance. The rotation limit element 720 contacts the band 710 to prevent further rotation and linear translation of the fixation mechanism 704 in the extension direction 716. For example, once the fixation mechanism 704 is rotated in the clockwise direction to extend the fixation mechanism 704 from the header body 700 by the extension distance, the rotation limit element 720 engages the band 710 and is prevented from being further rotated in the clockwise direction and displaced in the extension direction 716.

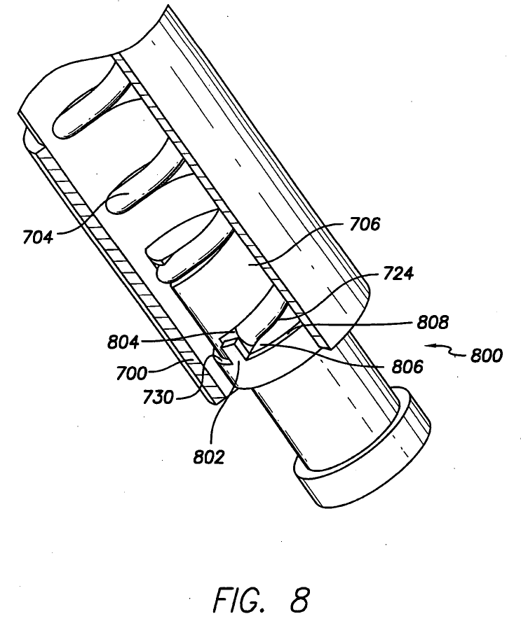

FIG. 8 is a partial sectional view of a proximal end 800 of the header body 700 in accordance with one embodiment. Several components of the header body 700 are not shown in FIG. 8 in order to more clearly demonstrate the spatial relationships of the header body 700, fixation mechanism 704 and shaft 706 in the illustrated embodiment. The header body 700 includes a proximal post 802 that protrudes from the shoulder 730 of the header body 700. The proximal post 802 extends from the shoulder 730 in a direction along the length of the header body 700.

The proximal post 802 is shaped and positioned to engage one or more of the shaft 706 and a proximal end 804 of the fixation mechanism 704 when the fixation mechanism 704 is retracted into the header body 700 by a predetermined retraction distance. For example, the channel 724 of the shaft 706 that receives the fixation mechanism 704 may extend around the shaft 706 such that the shaft 706 includes a rotation limit element 806 at or near the proximal end of the shaft 706. The rotation limit element 806 includes an edge of the shaft 706 that is located between the channel 724 and a rearward surface 808 of the shaft 706. The rearward surface 808 is a surface of the shaft 706 that may engage the shoulder 730 when the shaft 706 is retracted into the header body 700.

The fixation mechanism 704 may be positioned about the shaft 706 such that the proximal end 804 of the fixation mechanism 704 is located at or near the rotation limit element 806 of the shaft 706. In one embodiment, the proximal end 804 and rotation limit element 706 are approximately flush with respect to one another. Alternatively, the proximal end 804 of the fixation mechanism 704 may project past the rotation limit element 806 along the helical path of the channel 724. In another embodiment, the proximal end 804 of the fixation mechanism 704 may be recessed within the channel 724 such that the proximal end 804 does not project past the rotation limit element 806.

The shaft 706 and fixation mechanism 704 rotate as the shaft 706 and fixation mechanism 704 are retracted into the header body 700. For example, the counterclockwise rotation of the fixation mechanism 704 may cause the fixation mechanism 704 and shaft 706 to retract within the header body 700 toward the shoulder 730. Once the fixation mechanism 704 is retracted into the header body 700 by a predetermined retraction distance, the rotation limit element 806 of the shaft 706 and/or the proximal end 804 of the fixation mechanism 704 engage the proximal post 802. The proximal post 802 prevents additional rotation of the shaft 706 and the fixation mechanism 704.

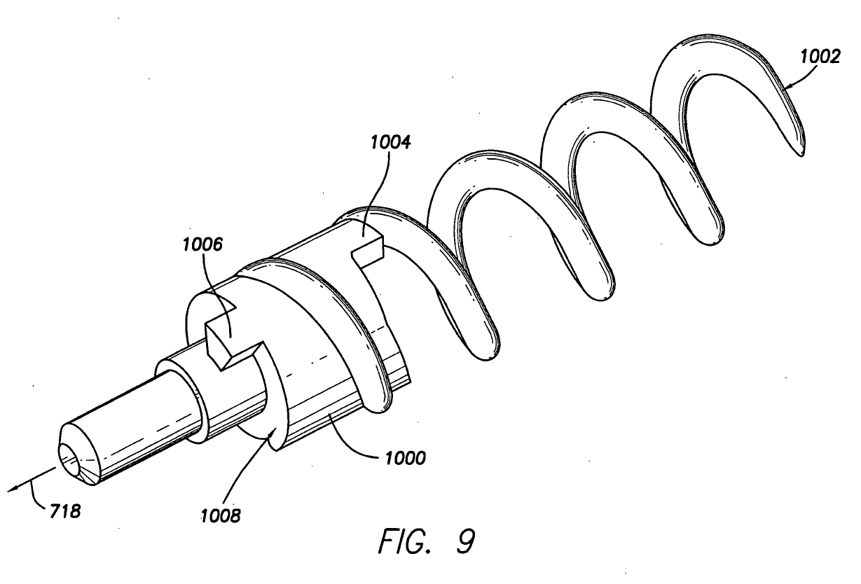

FIG. 9 is a perspective view of a rotatable shaft 1000 joined to a fixation mechanism 1002 in accordance with one embodiment. The shaft 1000 and fixation mechanism 1002 may be included in the header body 700 (shown in FIG. 7) in place of the shaft 706 (shown in FIG. 7) and the fixation mechanism 704 (shown in FIG. 7). The shaft 1000 includes a distal rotation limit element 1004 that is similar to the rotation limit element 720 (shown in FIG. 7). As described above, the distal rotation limit element 1004 engages the band 710 (shown in FIG. 7) to stop rotation of the shaft 1000 and fixation mechanism 1002 once the fixation mechanism 1002 has been extended by a predetermined extension distance. The shaft 1000 includes a proximal rotation limit element 1006 that projects along the length of the shaft 1000 from a rearward surface 1008 of the shaft 1000. The rearward surface 1008 faces away from the tip 726 (shown in FIG. 7) of the lead 702 (shown in FIG. 7) when the shaft 1000 is disposed in the header body 700. The proximal rotation limit element 1006 projects in the retraction direction 718 away from the rearward surface 1008.

The fixation mechanism 1002 is rotated to move the shaft 1000 and the fixation mechanism 1002 in the retraction direction 718. Once the fixation mechanism 1002 is retracted into the header body 700 (shown in FIG. 7) by a predetermined retraction distance, the proximal rotation limit element 1006 of the shaft 1000 engages the proximal post 802 (shown in FIG. 8) of the header body 700. The contact between the proximal rotation limit element 1006 and the proximal post 802 prevents further rotation of the shaft 1000 and the fixation mechanism 1002. For example, the proximal rotation limit element 1006 and proximal post 802 may stop additional rotation of the shaft 1000 and further travel of the shaft 1000 and fixation mechanism 1002 in the retraction direction 718.

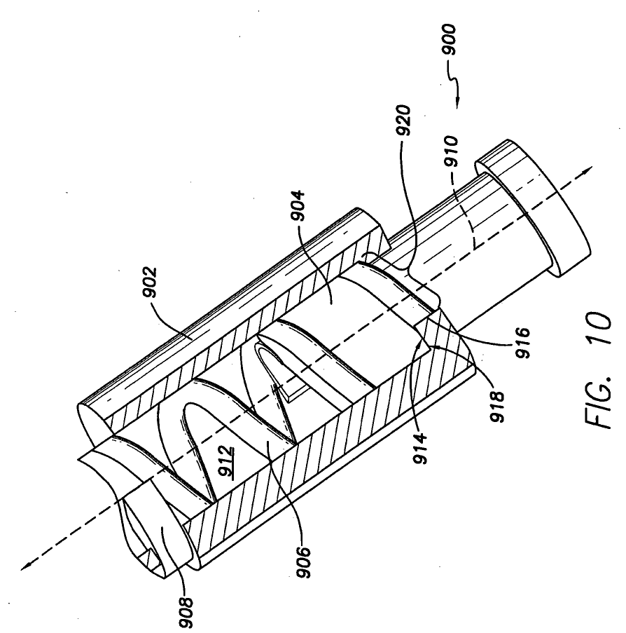

FIG. 10 is a partial sectional view of a proximal end 900 of a header body 902 in accordance with one embodiment. The header body 902 is joined to a lead (not shown) that is similar to the leads 102 (shown in FIG. 1) and 702 (shown in FIG. 2). The header body 902 is similar to the header bodies 222 (shown in FIG. 2) and 700 (shown in FIG. 7). For example, the header body 902 includes an interior chamber 912 that is similar to the interior chamber 724 (shown in FIG. 7). The inside surface of the header body 902 includes a shoulder 914 that is similar to the shoulder 730 (shown in FIG. 7) of the header body 700.

The header body 902 includes a rotatable shaft 904 joined to a fixation mechanism 906. The fixation mechanism 906 may be similar to the fixation mechanisms 212 (shown in FIG. 2) and 704 (shown in FIG. 7). The header body 902 includes a band 908 that is similar to the band 710 (shown in FIG. 7). Similar to the embodiments described above, the shaft 904 is rotated to rotate the fixation mechanism 906. As the fixation mechanism 906 rotates, the fixation mechanism 906 engages and slides along the band 908 to translate the rotary movement into linear displacement along a longitudinal axis 910 of the header body 902. The fixation mechanism 906 is located about the shaft 904 such that a proximal end 916 of the fixation mechanism 906 projects past a proximal end 918 of the shaft 904. Several additional components of the header body 902 are not shown in FIG. 10 in order to more clearly demonstrate the spatial relationships of the header body 902, fixation mechanism 906 and shaft 904 in the illustrated embodiment. For example, an electrical conductor (not shown) that is similar to the electrical conductors 318 (shown in FIG. 3) and 708 (shown in FIG. 7) may be coupled with the shaft 904.

The header body 902 includes a cavity 920 disposed in the shoulder 914 of the interior chamber 912. The cavity 920 defines a recess in the inner surface of the header body 902. The cavity 920 extends into the shoulder 914 along the length of the header body 902. As shown in FIG. 10, the cavity 920 may have a shape that approximately follows the helical path of the fixation mechanism 906 about the shaft 904. The cavity 920 is shaped and positioned to receive the proximal end 916 of the fixation mechanism 906 when the fixation mechanism 906 is retracted into the header body 902 by a predetermined retraction distance. For example, fixation mechanism 906 may be rotated by the shaft 904 such that the fixation mechanism 906 is retracted toward the shoulder 914 in the header body 902. The fixation mechanism 906 continues to be rotated and travel along the longitudinal axis 910 toward the shoulder 914 until the proximal end 916 of the fixation mechanism 906 is received into the cavity 920 and is prevented from further rotation. The cavity 920 prevents the fixation mechanism 906 from further rotation in the direction that retracts the fixation mechanism 906 into the header body 902.

One or more embodiments described herein provide an implantable lead that stops or impedes continued rotation of a rotatable shaft and a fixation mechanism coupled to the shaft once the fixation mechanism is extended from or retracted into the lead by a predetermined distance. Stopping the rotation of the shaft also limits the extension or retraction of the fixation mechanism from and into the lead. By preventing the continued rotation of the shaft once the fixation mechanism has been displaced by the predetermined distance, the leads described herein may stop an operator of the lead from over-torquing the fixation mechanism and damaging the shaft and/or fixation mechanism.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable lead comprising:
a lead body extending between a distal end and a proximal end, the lead body configured to be implanted in a patient;
a header body joined to the lead body at the distal end of the lead body;
a fixation mechanism disposed in the header body, the fixation mechanism extendable out of the header body for securing the header body to cardiac tissue of the patient;
a rotatable shaft provided in the header body, the shaft coupled to the fixation mechanism for translating rotational movement of the shaft into linear displacement of the fixation mechanism; and
a rotation limit element disposed in the header body, wherein the rotation limit element engages the header body once the shaft is linearly displaced by a predetermined distance with respect to the header body to prevent further rotation of the shaft and limit additional displacement of the fixation mechanism with respect to the header body.

2. The implantable lead of claim 1, wherein rotation of the shaft extends the fixation mechanism out of the header body toward the cardiac tissue and moves the rotation limit element in an extension direction with respect to the header body.

3. The implantable lead of claim 1, wherein the header body comprises an inwardly protruding post, the rotation limit element engaging the post to prevent further rotation of the shaft and prevent the fixation mechanism from being extended from the header body past a predetermined extension distance.

4. The implantable lead of claim 1, wherein the rotation limit element includes a radially protruding flange that engages the header body to prevent rotation of the shaft and the fixation mechanism with respect to the header body.

5. The implantable lead of claim 1, wherein the rotation limit element includes a tab protruding from the shaft along a length of the shaft.

6. The implantable lead of claim 1, wherein a proximal end of the fixation mechanism terminates proximate to a rearward surface of the shaft, the proximal end of the fixation mechanism and the rotation limit element engaging the header body to prevent further rotation of the shaft after the shaft is retracted into the header body by a predetermined retraction distance.

7. The implantable lead of claim 1, wherein a proximal end of the fixation mechanism terminates proximate to a rearward surface of the shaft and the header body comprises a cavity shaped to receive the proximal end of the fixation mechanism to prevent further rotation of the shaft once the shaft is retracted into the header body by a predetermined retraction distance.

8. The implantable lead of claim 1, wherein the rotation limit element is coupled to the shaft such that rotation of the shaft rotates the rotation limit element.

9. The implantable lead of claim 1, wherein the shaft and the rotation limit element include polarization features to align the rotation limit element with respect to the header body such that the rotation limit element engages the header body after the fixation mechanism is extended a predetermined extension distance from the header body.

10. The implantable lead of claim 1, wherein the fixation mechanism is electrically coupled with an implantable medical device, the fixation mechanism configured to provide a conductive pathway between the cardiac tissue and the implantable medical device.

11. An implantable lead comprising:
a lead body extending between a distal end and a proximal end, the lead body configured to be implanted in a patient;
a header body joined to the lead body at the distal end of the lead body;
a fixation mechanism disposed in the header body, the fixation mechanism extendable out of the header body for securing the header body to cardiac tissue of the patient; and
a rotatable shaft provided in the header body and coupled to the fixation mechanism for translating rotational movement of the shaft into linear displacement of the fixation mechanism, wherein the shaft engages the header body once the shaft is linearly displaced by a predetermined distance with respect to the header body to prevent further rotation of the shaft and to limit additional linear displacement of the fixation mechanism with respect to the header body.

12. The implantable lead of claim 11, wherein the shaft engages the header body to prevent further rotation of the shaft and limit retraction of the fixation mechanism into the header body past a predetermined retraction distance.

13. The implantable lead of claim 11, wherein the shaft comprises a radially projecting flange that engages the header body to prevent further rotation of the shaft when the fixation mechanism is retracted into the header body by a predetermined retraction distance.

14. The implantable lead of claim 11, wherein the shaft comprises a tab protruding from the shaft along a length of the shaft, the tab engaging the header body to prevent rotation of the shaft when the fixation mechanism is linearly displaced by the predetermined distance.

15. The implantable lead of claim 11, wherein a proximal end of the fixation mechanism terminates proximate to a rearward surface of the shaft, the proximal end of the fixation mechanism and the shaft engaging the header body to prevent further retraction of the shaft past a predetermined retraction distance.

16. The implantable lead of claim 11, wherein a proximal end of the fixation mechanism terminates proximate to a rearward surface of the shaft and the header body comprises a cavity shaped to receive the proximal end of the fixation mechanism to prevent further rotation of the shaft once the shaft is retracted into the header body by a predetermined retraction distance.

17. The implantable lead of claim 11, further comprising a rotation limit element coupled to the shaft, wherein the rotation limit element engages the header body once the shaft is linearly displaced by a predetermined extension distance with respect to the header body to prevent further rotation of the shaft and limit additional extension of the fixation mechanism from the header body.

18. The implantable lead of claim 11, wherein the fixation mechanism is electrically coupled with an implantable medical device, the fixation mechanism configured to provide a conductive pathway between the cardiac tissue and the implantable medical device.

19. An implantable lead comprising:
a lead body extending between a distal end and a proximal end, the lead body configured to be implanted in a patient;
a header body joined to the lead body at the distal end of the lead body;
a fixation mechanism disposed in the header body, the fixation mechanism configured to secure the header body to cardiac tissue of the patient; and
a rotatable shaft provided in the header body and coupled to the fixation mechanism for translating rotational movement of the shaft into linear displacement of the fixation mechanism to extend and retract the fixation mechanism with respect to the header body, wherein the shaft engages the header body once the shaft is linearly displaced by at least one of a predetermined extension distance and a predetermined retraction distance with respect to the header body to prevent further rotation of the shaft and limit additional displacement of the fixation mechanism with respect to the header body.

20. The implantable lead of claim 19, wherein the header body includes a plurality of inwardly protruding posts, the shaft engaging one of the posts to stop rotation of the shaft and the fixation mechanism when the fixation mechanism is extended from the header body to the extension distance, the shaft engaging another post to stop rotation of the shaft and the fixation mechanism when the fixation mechanism is retracted into the header body by the retraction distance.

21. The implantable lead of claim 19, further comprising a rotation limit element disposed in the header body, the rotation limit element engaging the header body once the shaft is linearly displaced by the extension distance with respect to the header body to prevent further rotation of the shaft and the fixation mechanism and limit additional extension of the fixation mechanism with respect to the header body.

22. The implantable lead of claim 19, wherein the shaft comprises a tab protruding from the shaft along a length of the shaft, the tab engaging the header body to prevent rotation of the shaft when the fixation mechanism is linearly displaced by the predetermined distance.

23. The implantable lead of claim 19, wherein a proximal end of the fixation mechanism terminates proximate to a rearward surface of the shaft, the proximal end of the fixation mechanism and the shaft engaging the header body to prevent further retraction of the shaft past a predetermined retraction distance.

24. The implantable lead of claim 19, wherein a proximal end of the fixation mechanism terminates proximate to a rearward surface of the shaft and the header body comprises a cavity shaped to receive the proximal end of the fixation mechanism to prevent further rotation of the shaft once the shaft is retracted into the header body by a predetermined retraction distance.

25. The implantable lead of claim 19, wherein the shaft includes a radially protruding flange that engages the header body.
